(19) United States
(12) Patent Application Publication
(10) Pub. No.: US 2014/0119502 A1
(43) Pub. Date: May 1, 2014

(54) RADIATION IMAGING APPARATUS AND METHOD OF OPERATING THE SAME

(71) Applicant: SAMSUNG ELECTRONICS CO., LTD., Suwon-si (KR)

(72) Inventor: Young-seok KANG, Suwon-si (KR)

(73) Assignee: SAMSUNG ELECTRONICS CO., LTD., Suwon-si (KR)

(21) Appl. No.: 13/858,335

(22) Filed: Apr. 8, 2013

(30) Foreign Application Priority Data
Nov. 1, 2012 (KR) 10-2012-0123097

(51) Int. Cl. A61B 6/03

(52) U.S. Cl. CPC A61B 6/032
USPC 378/19

(57) ABSTRACT

A method for operating an X-ray computed tomography (CT) apparatus includes obtaining a first projection data set by scanning an area of interest with X-rays at a first time; obtaining a second projection data set by scanning the area of interest with X-rays at a second time; obtaining at least one item of difference data which includes a difference between a first item of projection data from among the first projection data set and a corresponding item of projection data from among the second projection data set; and determining a rescanning of the area of interest by using X-rays based on information relating to the difference data.

### CROSS-REFERENCE TO PATENT APPLICATION

This application claims priority from Korean Patent Application No. 10-2012-0123097, filed on Nov. 1, 2012, in the Korean Intellectual Property Office, the disclosure of which is incorporated herein by reference in its entirety.

### BACKGROUND

1. Field

One or more exemplary embodiments relate to an radiation imaging apparatus and a method for operating the same.

2. Description of the Related Art

An X-ray computed tomography (CT) apparatus is a device which may be used for scanning an examination object, such as, for example, a physical body, by using a certain dose of X-rays, measuring the X-rays which pass through the examination object by using an X-ray detector, obtaining an X-ray absorption rate which is measured at various points of the examination object, and thus, using the obtained X-ray absorption rate to reconstruct an image. For this, the X-ray CT apparatus is required to perform X-ray photographing for two-dimensional (2D) images in order to obtain images of one point of the examination object from diverse angles. However, obtaining X-ray photographs of the examination object may be excessively time-consuming, and the examination object may be exposed to relatively large amounts of X-ray radiation.

### SUMMARY

One or more exemplary embodiments provide an radiation imaging apparatus for monitoring an examination object with X-rays which are supplied at a low dose, and a method for operating the same.

According to an aspect of one or more exemplary embodiments, there is provided a method for operating an radiation imaging apparatus, including obtaining a first projection data set by scanning an area of interest by with X-rays at a first time; obtaining a second projection data set by scanning the area of interest with X-rays at a second time; obtaining at least one item of difference data which includes a difference between a first item of projection data from among the first projection data set and a corresponding item of projection data from among the second projection data set; and determining whether to perform a rescanning of the area of interest with X-rays based on information relating to the difference data.

The first projection data set may be obtained by exposing the area of interest to X-rays at a first set of beam angles, and the second projection data set may be obtained by exposing the area of interest to X-rays at a second set of beam angles which have a phase difference of one of 0° and 180° with respect to the first set of beam angles.

The difference data may be obtained by using a contrast medium which flows into the area of interest.

An amount of the contrast medium may vary directly with a magnitude of the difference included in the difference data.

The information relating to the difference data may be expressed in units of a CT value.

If the information relating to the difference data has a value which is equal to or greater than a predetermined reference value, the rescanning of the area of interest may be determined to be terminated.

If the information relating to the difference data has a value which is less than a predetermined reference value, the rescanning of the area of interest may be determined to be performed.

The method for operating an radiation imaging apparatus may further include obtaining a third projection data set by rescanning the area of interest with X-rays at a third time; and determining whether to perform a further rescanning of the area of interest with X-rays based on at least one difference between a second item of projection data from among the first projection data set and a corresponding item of projection data from among the third projection data set.

The method for operating an X-ray CT apparatus may further include, if the information relating to the difference data has a value which is equal to or greater than a predetermined reference value, scanning an examination area.

The method for operating an X-ray CT apparatus may further include, if the information relating to the difference data has a value which is equal to or greater than a predetermined reference value, checking inflow of a contrast medium into the area of interest.

If the information relating to the difference data relates to at least two items of the difference data, the determining whether to perform rescanning of the area of interest may be based on a comparison of each of the at least two items of the difference data to a predetermined reference value.

Additionally, if the information relating to the difference data relates to at least two items of the difference data, the determining whether to perform rescanning of the area of interest may be based on a comparison of an average of values included in the information relating to the difference data with a predetermined reference value.

The scanning of the area of interest with X-rays at the first time may be performed by exposing the area of interest to X-rays from N beam angles, wherein N is a natural number.

Among the N beam angles, a phase difference between neighboring beam angles may be greater than 0° and less than 180°.

Among the N beam angles, a phase difference between neighboring beam angles may be equal to 180°/N.

The first projection data set may be obtained before a contrast medium flows into the area of interest.

According to another aspect of one or more exemplary embodiments, there is provided an radiation imaging apparatus including: an X-ray scanner which scans an examination object with X-rays; an X-ray detector which discontinuously detects X-rays which pass through the examination object; a processor which obtains a plurality of items of projection data which relate to an area of interest of the examination object, by using the detected X-rays; and a controller which determines whether to perform a rescanning of the area of interest by using the X-rays based on at least one difference which is computed by using the plurality of items of projection data.

The X-ray scanner discontinuously may scan the examination object with the X-rays.

The X-ray scanner may expose the area of interest to the X-rays at N beam angles, wherein N is a natural number.

Among the N beam angles, a phase difference between neighboring beam angles may be equal to 180°/N.

The X-ray scanner may include an X-ray generator which generates the X-rays; and a collimator which obstructs a passage of X-rays into areas which are outside of the area of interest, from among the X-rays generated by the X-ray generator.

The X-ray generator may discontinuously generate the X-rays.

The collimator may discontinuously obstruct the passage of the X-rays into the area of interest.

The plurality of items of projection data may be obtained by exposing the area of interest to X-rays at beam angles which are the same or have a phase difference of 180°.

If the at least one difference which is computed by using the plurality of items of projection data is equal to or greater than a predetermined reference value, the controller may control the X-ray scanner to terminate the rescanning of the area of interest.

### BRIEF DESCRIPTION OF THE DRAWINGS

The above and other features and advantages of the present inventive concept will become more apparent by describing in detail exemplary embodiments thereof with reference to the attached drawings in which:

FIG. 1 is a diagram which illustrates a partial appearance of an radiation imaging apparatus, according to an exemplary embodiment;

FIG. 2 is a block diagram which illustrates the radiation imaging apparatus of FIG. 1, according to an exemplary embodiment;

FIG. 3A, FIG. 3B, and FIG. 3C are diagrams which illustrate a beam angle of an X-ray scanner according to the number of exposure times, according to an exemplary embodiment.

FIG. 4A is a block diagram which illustrates the X-ray scanner of FIG. 2, according to an exemplary embodiment;

FIG. 4B is another block diagram which illustrates the X-ray scanner of FIG. 2, according to another exemplary embodiment;

FIG. 5 is a block diagram which illustrates a signal processor of the radiation imaging apparatus of FIG. 2; and

FIG. 6 is a flowchart which illustrates a method for operating a signal processor in a pre-photographing mode, according to an exemplary embodiment.

### DETAILED DESCRIPTION

Hereinafter, an radiation imaging apparatus and a method for operating the same, according to one or more exemplary embodiments, will be described in detail by explaining the exemplary embodiments with reference to the attached drawings. Like reference numerals in the drawings denote like elements, and thus their description will be omitted.

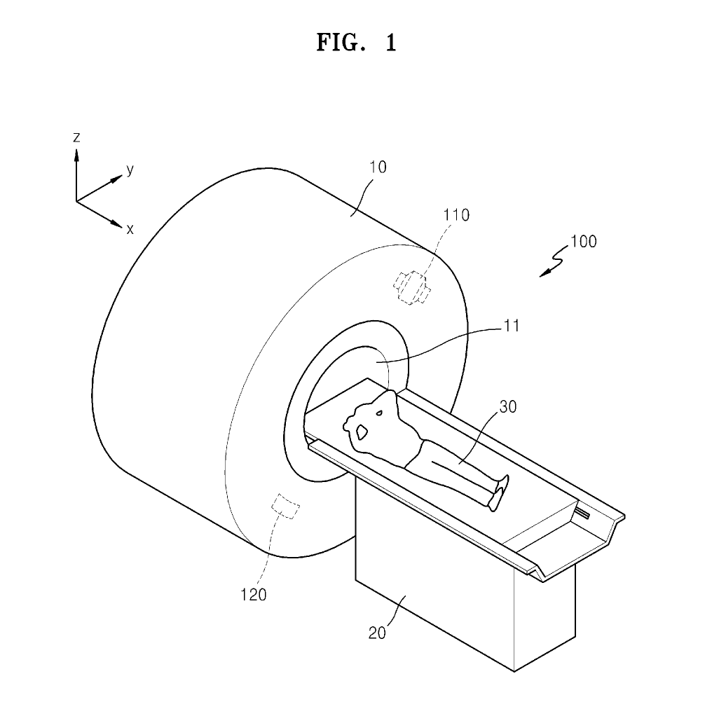

FIG. 1 is a diagram which illustrates a partial appearance of an radiation imaging apparatus 100, according to an exemplary embodiment. The radiation imaging apparatus 100 may be an X ray CT apparatus. As illustrated in FIG. 1, the radiation imaging apparatus may include a gantry 10 and an examination table 20. Referring to FIG. 1, the gantry 10 includes an opening 11, which is cylinder-shaped, which receives an examination object 30. The gantry 10 may also include an X-ray scanner 110 which scans X-rays and an X-ray detector 120 which detects X-rays which pass through an examination object. The X-ray scanner 110 is disposed to face the X-ray detector 120 with the examination object 30 therebetween at a center of a particular area which surrounds the opening 11 of the gantry 10. For example, the X-ray scanner 110 and the X-ray detector 120 are included in the gantry 10, and are arranged to scan and detect an x-ray beam which is incident in a vertical direction.

The gantry 10 is driven by a gantry driver (not illustrated) which rotates around the examination object 30 in 360 degrees or in a predetermined number of degrees. Thus, the examination object 30 may be photographed from diverse angles by using the X-ray scanner 110 and the X-ray detector 120. Additionally, the gantry driver may be moved forward and backward in a horizontal direction which is along an X-axis. Thus, an area of the examination object 30 that is to be photographed on the examination table 20 may be placed at a central portion inside the gantry 10. The gantry driver may be included in the gantry 10 or disposed outside the gantry 10.

The examination table 20 is formed as a bed which has a particular size, on which a patient, who is the examination object 30, is laid and still. A particular area of the examination table 20 may include an examination table driver (not illustrated) which transports the examination table 20 into the opening 11 at a central portion of the gantry 10. The examination table 20 may be driven forward and backward in a horizontal direction so that the area of the patient to be photographed is located within the central portion of the gantry 10. The driver of the examination table 20 may drive the examination table 20 upwards and downwards, which is along a Z-axis, or to the left and right, which is along a Y-axis, based on a physical size and area of the patient to be photographed, so as to enable the apparatus 100 to obtain a clear image.

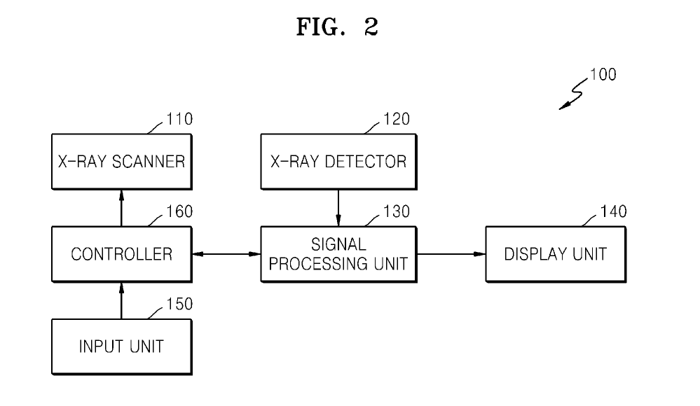

FIG. 2 is a block diagram which illustrates the radiation imaging apparatus 100 of FIG. 1. The block diagram of the radiation imaging apparatus 100 specifies units which are necessary for obtaining an image by performing X-ray scanning.

As illustrated in FIG. 2, the radiation imaging apparatus 100 may include the X-ray scanner 110 which scans the examination object 30 with X-rays, the X-ray detector 120 which detects X-rays which pass through the examination object 30, a signal processor 130 which obtains an image by using the X-rays detected by the X-ray detector 120, a display 140 which displays the obtained image, an input unit 150 which receives an input of a user command, and a controller 160 which controls all operations of the radiation imaging apparatus 100.

The X-ray scanner 110 generates X-rays in order to scan a patient who is the examination object 30. The X-ray scanner 110 may scan the examination object 30 with X-rays in a form of a sector.

Modes of the radiation imaging apparatus, according to an exemplary embodiment, are classified into a main photographing mode and a pre-photographing mode. In the main photographing mode, an examination area of the examination object 30 is photographed. In the pre-photographing mode, an area of interest is photographed so as to check the inflow of a contrast medium into the examination area. The examination area includes an area which relates to verifying any bodily abnormality, if the examination object 30 is a person, by displaying a heart, a lung, and/or a head of the person in the display 140. The area of interest includes an area which relates to monitoring the inflow of a contrast medium in the examination area prior to X-ray photographing of the examination area. If the examination area is a heart, the area of interest may include arteries in a chest where blood flows to the heart.

For X-ray photographing, a contrast medium may be injected into the examination object 30 in order to obtain a clearer image of an examination area of the examination object 30. The contrast medium flows into the examination area via blood vessels. The area of interest is photographed to identify a time point at which the contrast medium flows into the area of interest. As the area of interest is photographed only to monitor the inflow of the contrast medium, a lower resolution of an image thereof, as compared with a resolution of an image of the examination area, would be acceptable. Accordingly, several parameters associated with the main photographing mode and the pre-photographing mode may be different in terms of X-ray dose, the number of exposures, and a beam angle per one rotation of the gantry 10.

Scanning patterns of the X-ray scanner 110 include, for example, a conventional scan which is an axial scan, a helical scan, a variable pitch helical scan, and a helical shuttle scan. The conventional scan refers to a scanning method for obtaining projection data by rotating the X-ray scanner 110 and the X-ray detector 120 whenever the examination table 20 is moved along an X axis within a predetermined space. The helical scan refers to a photographing method for obtaining projection data by moving the examination table 20 at a constant speed while rotating the X-ray scanner 110 and the X-ray detector 120. The variable pitch helical scan, similar to the helical scan, refers to a photographing method for obtaining projection data by varying a speed of motion of the examination table 20 while rotating the X-ray scanner 110 and the X-ray detector 120. The helical shuttle scan, similar to the helical scan, refers to a scanning method for obtaining projection data by accelerating or decelerating the motion of the examination table 20 so as to move forward and backward along an X-axis, while rotating the X-ray scanner 110 and the X-ray detector 120.

At least one pattern from among the scanning patterns may be applied to the main photographing mode. The pre-photographing mode may employ the conventional scan pattern, but is not limited thereto. Different scan patterns may be also used in the pre-photographing mode. However, the conventional scan pattern is described for convenience of description.

In particular, in the pre-photographing mode, the X-ray scanner 110 rotates around the examination object 30 in 360 degrees or in a predetermined number of degrees to discontinuously scan an area of interest of the examination object 30 with X-rays. More particularly, the X-ray scanner 110 may repeatedly perform X-ray exposures and interruption of exposures of the area of interest. For example, the X-ray scanner 110 may rotate and expose the area of interest to X-rays at N beam angles, where N is a natural number which is equal to or greater than 1, and the X-ray scanner 110 does not expose the area of interest to the X-rays at other beam angles. It is desirable to set the N beam angles to respectively obtain different projection data with regard to the area of interest.

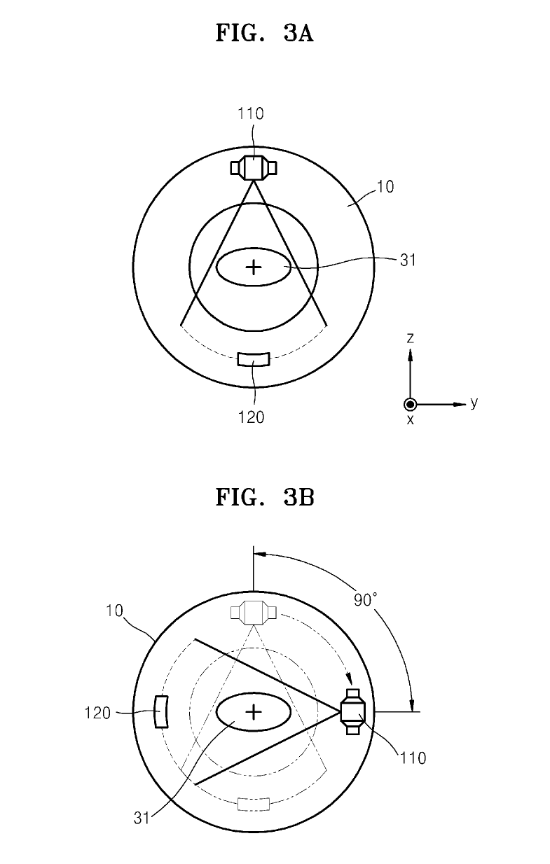

FIG. 3A, FIG. 3B, and FIG. 3C are diagrams which illustrate a beam angle of the X-ray scanner 110 according to the number of exposures, according to an exemplary embodiment. The beam angle may be defined as an angle of tilt with respect to a Z-axis, which is vertical with respect to a length and a width of the examination object 30.

In order to expose an area of interest 31 to X-rays once, as illustrated in FIG. 3A, the X-ray scanner 110 may expose the area of interest 31 to the X-rays along the Z-axis, which is oriented at a beam angle of 0°.

In order to expose the area of interest 31 to X-rays twice, the X-ray scanner 110 may expose the area of interest 31 to the X-rays at two beam angles at which different projection data may be respectively obtained. For example, as illustrated in FIG. 3B, the X-ray scanner 110 may expose the area of interest 31 to X-rays at a beam angle of 0°, which coincides with the Z-axis, and then, at a beam angle of 90°, which is tilted 90° from the Z-axis. Of course, the X-ray scanner 110 may expose the area of interest 31 to the X-rays at beam angles other than 90°. For example, the X-ray scanner 110 may expose the area of interest 31 to the X-rays at a beam angle of 45° or 120°. However, it is not desirable to employ 0° and 180° as beam angles, as projection data which respectively correspond to beam angles of 0° and 180° are in a reverse relationship.

Additionally, in order to expose the area of interest 31 to X-rays three times, the X-ray scanner 110 may expose the area of interest 31 to X-rays at three beam angles at which different projection data may be respectively obtained. For example, as illustrated in FIG. 3C, the X-ray scanner 110 may expose the area of interest 31 to X-rays at a beam angles of 0°, 60°, and 120°. However, the beam angles are presented only for convenience of description, and are not limited thereto. Exposures may also be performed at beam angles which are not in a reverse relationship. More desirably, the X-ray scanner 110 may expose the area of interest 31 to the X-rays at beam angles which allow the apparatus 100 to obtain mutually compensated projection data. For example, when exposures are performed at N beam angles, it is desirable to set a phase difference between neighboring beam angles to be equal to 180°/N.

As such, the X-ray scanner 110 may perform X-ray exposures only at specific beam angles, except for other beam angles. As the pre-photographing mode is performed at a relatively low X-ray dose, radiation exposure to an examination object may be reduced.

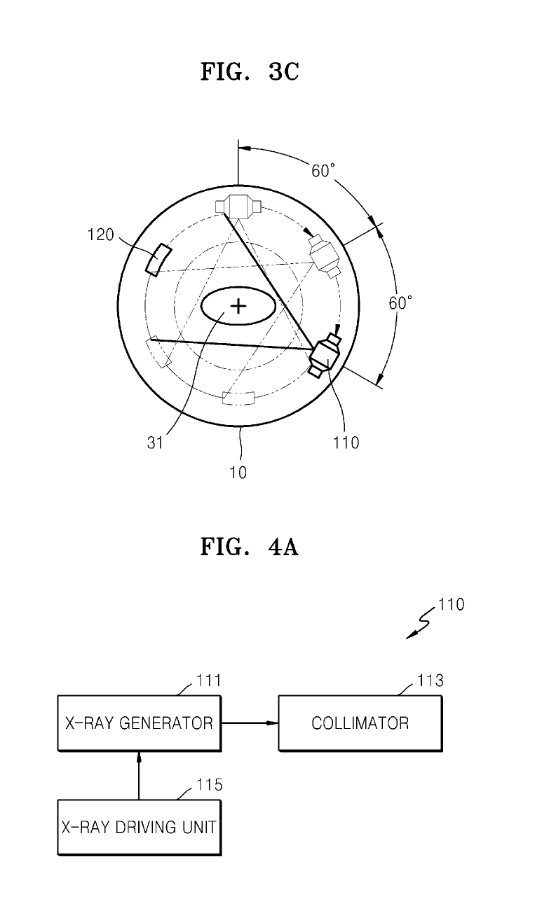

Next, a configuration relating to a discontinuous X-ray scanning which is performed by the X-ray scanner 110 is described. FIG. 4A is a block diagram which illustrates the X-ray scanner 110 of FIG. 2, according to an exemplary embodiment, and FIG. 4B is another block diagram which illustrates the X-ray scanner 110 of FIG. 2, according to another exemplary embodiment. As illustrated in FIGS. 4A and 4B, the X-ray scanner 110 may include an X-ray generator 111 which generates X-rays and a collimator 113 which obstructs generated X-rays. The collimator 113 obstructs passage of X-rays into areas which are outside of an area of interest, from among X-rays generated by the X-ray generator 111.

The X-ray scanner 110 may also expose an area of interest to X-rays only at a specific beam angle. For this, the X-ray generator 111 may discontinuously generate X-rays, or the collimator 113 may discontinuously obstruct the passage of the X-rays into the area of interest. For example, as illustrated in FIG. 4A, the X-ray scanner 110 may include an X-ray driver 115 which drives the X-ray generator 111. Otherwise, as illustrated in FIG. 4B, the X-ray scanner 110a may include a collimator driver 117 for controlling the collimator 113 so as to expose an area of interest to X-rays only at a specific beam angle, and thus, obstruct passage of X-rays into the area of interest at other beam angles.

The X-ray scanner 120 of FIG. 2 is synchronized with the X-ray scanner 110 in order to discontinuously detect X-rays and in order to convert the detected X-rays into an electrical signal. The X-ray detector 120 may be formed by using a set of a plurality of cells, and each cell converts the detected X-rays into an electrical signal. A flat panel detector may be used as the X-ray detector 120.

Additionally, the signal processor 130 receives an electrical signal which corresponds to the X-rays detected by the X-ray detector 120, and thus, obtains projection data or images of the area of interest or an examination area. A method for operating the signal processor 130 may vary according to whether the mode of the radiation imaging apparatus 100 is set to the pre-photographing mode or the main photographing mode.

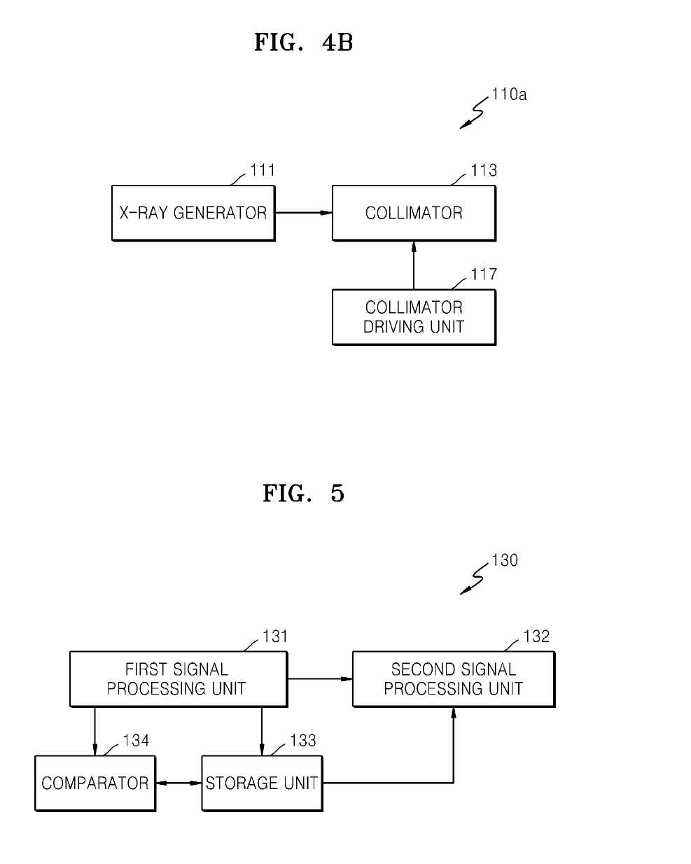

FIG. 5 is a block diagram which illustrates the signal processor 130 of the radiation imaging apparatus 100 of FIG. 2. As illustrated in FIG. 5, the signal processor 130 includes a first signal processor 131 which obtains projection data by pre-processing an electrical signal which corresponds to detected X-rays, a second signal processor 132 which obtains an image by reconstructing the projection data obtained according to beam angles, a storage 133 which stores the projection data, and a comparator 134 which compares the projection data to reference data, such as, for example, predetermined reference values, and which transmits a result of the comparison to a controller.

If the radiation imaging apparatus 100 operates in the main photographing mode, the first signal processor 131 and the second signal processor 132 in the signal processor 130 may operate. For example, the first signal processor 131 pre-processes an electrical signal which corresponds to detected X-rays and obtains projection data. The pre-processing may include at least one of offset compensation, algebraic transformation, X-ray dose compensation, sensitivity compensation, and beam hardening compensation.

The second signal processor 132 obtains an image by reconstructing a plurality of items of projection data. For example, the second signal processor 132 may obtain a two-dimensional (2D) tomographic image by reconstructing a plurality of items of projection data. The second signal processor 132 also may obtain a stereoscopic three-dimensional (3D) image by reconstructing a plurality of 2D tomographic images in an X-axis direction. The 2D tomographic images or the stereoscopic 3D images may be displayed on a display 140. Exemplary methods for displaying a stereoscopic 3D image include a 3D volume rendering image display method, a 3D maximum intensity projection (MIP) image display method, a multi-planar reformat (MPR) image display method, and a 3D reprojection data display method. The image display method may vary according to diagnostic purposes. As obtaining of 2D tomographic images and stereoscopic 3D images is well known by those skilled in the art, a detailed description thereof is not provided.

If the radiation imaging apparatus 100 is in the pre-photographing mode, the first signal processor 131 and the comparator 134 in the signal processor 130 operate. Hereinafter, a method for operating the radiation imaging apparatus in the pre-photographing mode is described in detail.

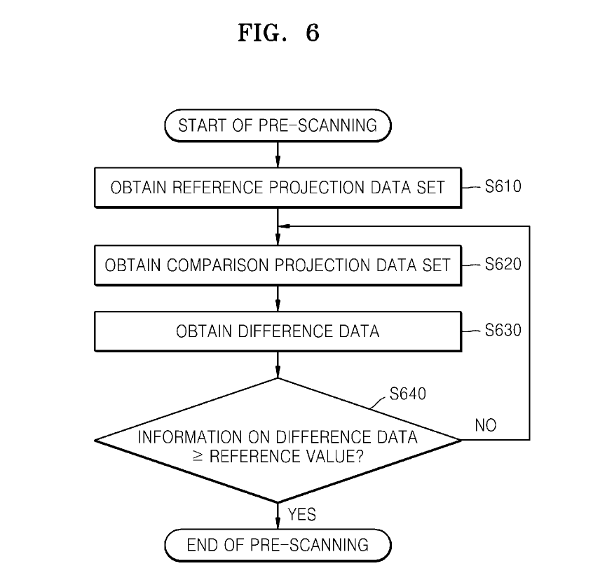

FIG. 6 is a flowchart which illustrates a method for operating the signal processor 130 in a pre-photographing mode, according to an exemplary embodiment. In operation S610, when a pre-photographing mode is set, the first signal processor 131 obtains a reference projection data set.

In particular, the X-ray scanner 110 may discontinuously scan an area of interest with X-rays. When the gantry 10 rotates once, the X-ray scanner 110 exposes an area of interest at least once, although the number of exposures in the pre-photographing mode may be smaller than the number of exposures in a main photographing mode. The X-ray detector 120 is synchronized with the X-ray scanner 110 in order to detect X-rays which have passed through the area of interest. The X-ray scanner 120 detects the generated X-rays for each beam angle, obtains an electrical signal from the detected X-rays, and thus, transmits the obtained electrical signal to the signal processor 130. Additionally, the first signal processor 131 may obtain projection data which corresponds to the X-rays for each beam angle.

A projection data set may include projection data which is obtained when the gantry 10 rotates once. When the gantry 10 rotates once and if the X-ray scanner 110 exposes the area of interest to X-rays at three beam angles, the first signal processor 131 may obtain one projection data set which includes three items of projection data which relate to the area of interest. Additionally, a reference projection data set includes a projection data set which is obtained during a time at which a contrast medium does not flow into the area of interest (hereinafter, referred to as a “first time”), prior to or immediately after the contrast medium is injected into an examination object.

Additionally, in operation S620, the first signal processor 131 obtains a comparison projection data set. When the gantry 10 rotates for a second time, the X-ray scanner 110 may expose the area of interest to X-rays at least once or more. The X-ray detector 120 is synchronized with the X-ray scanner 110 in order to detect X-rays which have passed through the area of interest. Then, the X-ray scanner 120 transmits a result obtained by using the detected X-rays to the signal processor 130. Additionally, the first signal processor 131 obtains a projection data set which corresponds to X-rays for each beam angle. A comparison projection data set includes projection data which is to be compared to the reference projection data set. The comparison projection data set represents projection data which is obtained during a time after the reference projection data set is obtained (hereinafter, referred to as a “second time”).

In operation S630, the comparator 134 obtains difference data which includes at least one difference between at least a first item of projection data from among the reference projection data set and a corresponding item of data from among the comparison projection data set. The corresponding items of projection data refer to respective data included in each of the reference projection data set and the comparison projection data set which is obtained by exposing the area of interest to X-rays at beam angles which are the same or have a phase difference of 180°. For example, the comparator 134 may compare reference projection data obtained by exposure to X-rays at a beam angle of 90° at the first time to comparison projection data obtained by exposure to X-rays at a beam angle of 90° at the second time. If exposures are performed at a plurality of beam angles, a plurality of items of difference data are obtained.

The comparator 134 compares information relating to the difference data to a predetermined reference value and transmits a result of the comparison to the controller 160. The controller 160 determines whether to perform a rescanning of the area of interest based on the result of the comparison. The reference value may vary based on types of contrast media or examination areas. The comparator 134 may convert the difference data into quantities having units of a CT value, and compare the CT value of the converted difference data to the reference value. The CT value is a unit for a density of X-ray dose.

In operation S640, a determination is made regarding whether the information relating to the difference data has a value which is greater than or equal to the predetermined reference value. As indicated by a “YES” determination with respect to operation S640, if the information relating to the difference data has a value which is equal to or greater than the reference value, the controller 160 terminates the pre-photographing mode and, after a predetermined time elapses, performs the main photographing mode. When a predetermined time elapses after the contrast medium flows into the area of interest, the contrast medium flows into the examination area. Then, the controller 160 controls an examination table driver to dispose the examination area at a central portion of the gantry 10 so that the examination area may be photographed after a predetermined time elapses. Otherwise, if the information relating to the difference data has a value which is equal to or greater than the reference value, the controller 160 may re-check the inflow of the contrast medium into the area of interest. For example, the controller 160 may control each component to obtain a tomographic image of the area of interest and re-check the inflow of the contrast medium into the area of interest by using a CT value of the tomographic image.

As indicated by a “NO” determination with respect to operation S640, if the information relating to the difference data has a value which is lower than the reference value, the controller 160 continues to perform the pre-photographing mode. When the information relating to the difference data has a value which is lower than the reference value, the information thusly indicates that a sufficient amount of the contrast medium has not yet flowed into the area of interest. Therefore, the controller 160 repeats operations S610 through S640. In particular, the X-ray scanner 110 re-scans the area of interest at a third time. The X-ray detector 120 detects the scanned X-rays and transmits a result of the detected scanned X-rays to the first signal processor 131. The first signal processor 131 obtains a projection data set. The comparator 134 obtains difference data, which includes at least one difference between at least a first item of projection data from among a reference projection data set and a corresponding item of projection data from among a comparison projection data set which are obtained at the third time. Then, the comparator 134 compares information relating to the obtained difference data to the reference value and transmits a result of the comparison to the controller 160.

If there are several items of difference data, the comparator 134 may compare each item of information relating to the difference data or an average of values included in the information relating to the difference data to the reference value. If there is a relatively large number of items of difference data, it is desirable to compare the average of the values included in the information relating to the difference data to the reference value. However, if there is a relatively small number of items of difference data, it is desirable to compare each item of information relating to the difference data to the reference value. Thus, the controller 160 may perform the pre-photographing mode until each item of information relating to the difference data or an average of the values included in the information relating to the difference data reaches the reference value.

According to an exemplary embodiment, a comparison is made between the projection data, which is obtained before the contrast medium flows into the area of interest, and the reference data, but is not limited thereto. For example, difference data may be obtained by using a first set of projection data which is currently obtained and a second set of projection data which was obtained shortly before the acquisition of the first set of projection data. Rescanning of the area of interest may be determined by checking whether a sum of CT values of each item of difference data exceeds the reference value.

As such, because inflow of a contrast medium into the area of interest is determined based on the CT values relating to the difference values, it is simpler to calculate the CT values relating to the difference values than to obtain CT values relating to an entirety of the projection data.

The image processing method in the pre-photographing mode of the signal processor 130 can be written as computer programs and can be implemented in general-use digital computers that execute the programs using a transitory or non-transitory computer-readable recording medium. Examples of the non-transitory computer-readable recording medium include magnetic storage media (e.g., read-only memory (ROM), floppy disks, hard disks, and any other suitable magnetic storage medium), and storage media such as optical recording media (e.g., compact disk-ROM (CD-ROMs) or digital versatile disks (DVDs)).

The radiation imaging apparatus 100 and the method for operating the same, according to the exemplary embodiments, may be monitored by using X-rays at a relatively low dose. Therefore, unnecessary radiation exposure of an examination object to X-ray radiation may be reduced.

Additionally, X-ray scanning is performed only at specific beam angles and projection data is obtained as a result of the X-ray scanning. Therefore, calculations for monitoring may be simplified.

As the present inventive concept allows for various changes and numerous embodiments, particular exemplary embodiments will be illustrated in the drawings and described in detail in the written description. However, this is not intended to limit the present inventive concept to particular modes of practice, and it is to be appreciated that all changes, equivalents, and substitutes that do not depart from the spirit and technical scope of the present disclosure are encompassed in the present inventive concept.

What is claimed is:

1. A method for operating an radiation imaging apparatus, the method comprising:
obtaining a first projection data set by scanning an area of interest with X-rays at a first time;
obtaining a second projection data set by scanning the area of interest with X-rays at a second time;
obtaining at least one item of difference data which comprises a difference between a first item of projection data from among the first projection data set and a corresponding item of projection data from among the second projection data set; and
determining whether to perform a rescanning of the area of interest with X-rays based on information relating to the difference data.

2. The method of claim 1, wherein the first item of projection data from among the first projection data is obtained by exposing the area of interest to X-rays at a first set of beam angles, and the corresponding item of projection data from among the second projection data set is obtained by exposing the area of interest to X-rays at a second set of beam angles which have a phase difference of one of 0° and 180° with respect to the first set of beam angles.

3. The method of claim 1, wherein the difference data is obtained by using a contrast medium which flows into the area of interest.

4. The method of claim 3, wherein an amount of the contrast medium varies directly with a magnitude of the difference included in the difference data.

5. The method of claim 1, wherein the information relating to the difference data is expressed in units of a CT value.

6. The method of claim 1, wherein, if the information relating to the difference data has a value which is equal to or greater than a predetermined reference value, the rescanning of the area of interest is determined to be terminated.

7. The method of claim 1, wherein, if the information relating to the difference data has a value which is less than a predetermined reference value, the rescanning of the area of interest is determined to be performed.

8. The method of claim 7, further comprising:
obtaining a third projection data set by rescanning the area of interest with X-rays at a third time; and
determining whether to perform a further rescanning of the area of interest with X-rays based on at least one difference between the first item of projection data from among the first projection data set and a corresponding item of projection data from among the third projection data set.

9. The method of claim 1, further comprising, if the information relating to the difference data has a value which is equal to or greater than a predetermined reference value, scanning an examination area.

10. The method of claim 1, further comprising, if the information relating to the difference data has a value which is equal to or greater than a predetermined reference value, checking an inflow of a contrast medium into the area of interest.

11. The method of claim 1, wherein, if the information relating to the difference data relates to at least two items of the difference data, the determining whether to perform rescanning of the area of interest is based on a comparison of each of the at least two items of the difference data to a predetermined reference value.

12. The method of claim 1, wherein, if the information relating to the difference data relates to at least two items of the difference data, the determining whether to perform rescanning of the area of interest is based on a comparison of an average of values included in the information relating to the difference data with a predetermined reference value.

13. The method of claim 1, wherein the scanning of the area of interest with X-rays is performed by exposing the area of interest to X-rays from N beam angles, wherein N is a natural number.

14. The method of claim 13, wherein among the N beam angles, a phase difference between neighboring beam angles is greater than 0° and less than 180°.

15. The method of claim 13, wherein among the N beam angles, a phase difference between neighboring beam angles is equal to 180°/N.

16. The method of claim 1, wherein the first projection data set is obtained before a contrast medium flows into the area of interest.

17. An radiation imaging apparatus comprising:
an X-ray scanner which scans an examination object with X-rays;
an X-ray detector which discontinuously detects X-rays which pass through the examination object;
a processor which obtains a plurality of items of projection data which relate to an area of interest of the examination object, by using the detected X-rays; and
a controller which determines whether to perform a rescanning of the area of interest by using the X-rays based on at least one difference which is computed by using the plurality of items of projection data.

18. The X-ray CT apparatus of claim 17, wherein the X-ray scanner discontinuously scans the examination object with the X-rays.

19. The X-ray CT apparatus of claim 17, wherein the X-ray scanner exposes the area of interest to the X-rays at N beam angles, wherein N is a natural number.

20. The X-ray CT apparatus of claim 19, wherein among the N beam angles, a phase difference between neighboring beam angles is equal to 180°/N.

21. The X-ray CT apparatus of claim 17, wherein the X-ray scanner comprises:
an X-ray generator which generates the X-rays; and
a collimator which obstructs a passage of X-rays into areas which are outside of the area of interest, from among the X-rays generated by the X-ray generator.

22. The X-ray CT apparatus of claim 21, wherein the X-ray generator discontinuously generates the X-rays.

23. The X-ray CT apparatus of claim 21, wherein the collimator discontinuously obstructs the passage of the X-rays into the area of interest.

24. The X-ray CT apparatus of claim 17, wherein the plurality of items of projection data are obtained by exposing the area of interest to X-rays at beam angles which are the same or have a phase difference of 180°.

25. The X-ray CT apparatus of claim 17, wherein, if the at least one difference which is computed by using the plurality of items of projection data is equal to or greater than a predetermined reference value, the controller controls the X-ray scanner to terminate the rescanning of the area of interest.

26. A method for operating an X-ray computed tomography (CT) apparatus, the method comprising:
obtaining a first projection data set by scanning an area of interest with X-rays from a first set of beam angles at a first time;
using a contrast medium which flows into the area of interest;
obtaining a second projection data set by scanning the area of interest with X-rays from a second set of beam angles at a second time, wherein the second set of beam angles has a phase difference of one of 0° and 180° with respect to the first set of beam angles;
computing at least one difference between a first item of projection data from among the first projection data set and a corresponding item of projection data from among the second projection data set; and
determining whether to perform a rescanning of the area of interest with X-rays based on the computed at least one difference.

27. The method of claim 26, wherein the first set of beam angles comprises at least a first beam angle and a second beam angle which has a phase difference of between 45° and 120° with respect to the first beam angle.

28. The method of claim 26, wherein the determining whether to perform the rescanning of the area of interest comprises comparing the computed at least one difference with a predetermined reference value and determining whether to perform the rescanning of the area of interest based on a result of the comparing.

29. The method of claim 26, wherein when a determination is made to perform the rescanning of the area of interest, the method further comprises obtaining a third projection data set by rescanning the area of interest with X-rays from the second set of beam angles at a third time;
computing at least a second difference between a first item of projection data from among the first projection data set and a corresponding item of projection data from among the third projection data set; and
determining whether to perform a further rescanning of the area of interest with X-rays based on the computed at least the second difference.

30. A non-transitory computer readable storage medium having recorded thereon a program which is executable by a computer for performing a method for operating an X-ray computed tomography (CT) apparatus, the method comprising:
obtaining a first projection data set by controlling the apparatus to scan an area of interest with X-rays at a first time;
obtaining a second projection data set by controlling the apparatus to scan the area of interest with X-rays at a second time;
obtaining at least one item of difference data which comprises a difference between a first item of projection data from among the first projection data set and a corresponding item of projection data from among the second projection data set; and
determining whether to control the apparatus to perform a rescanning of the area of interest with X-rays based on information relating to the difference data.
